(19) United States
(12) Patent Application Publication
(10) Pub. No.: US 2026/0022538 A1
(43) Pub. Date: Jan. 22, 2026

(54) SYSTEM INCLUDING WORK MACHINE, CONTROL METHOD, AND WORK MACHINE

(71) Applicants: KOMATSU LTD., Minato-ku, Tokyo (JP); The University of Tokyo, Bunkyo-ku, Tokyo (JP)

(72) Inventors: Kazuhiro MURATA, Minato-ku, Tokyo (JP); Kazushi NAKATA, Minato-ku, Tokyo (JP); Naoki TSUJIMOTO, Minato-ku, Tokyo (JP); Masayuki NAKAO, Bunkyo-ku, Tokyo (JP); Kazutaka UEDA, Bunkyo-ku, Tokyo (JP)

(73) Assignees: KOMATSU LTD., Minato-ku, Tokyo (JP); The University of Tokyo, Bunkyo-ku, Tokyo (JP)

(21) Appl. No.: 18/995,421

(22) PCT Filed: Jul. 26, 2023
(86) PCT No.: PCT/JP2023/027345

§ 371 Date: Jan. 16, 2025

(30) Foreign Application Priority Data
Aug. 23, 2022 (JP) 2022-132583

(51) Int. Cl. E02F 9/24; A61B 5/18; E02F 9/26; G08B 21/18

(52) U.S. Cl. CPC E02F 9/24; A61B 5/18; E02F 9/26; G08B 21/18

(57) ABSTRACT

A system capable of giving a notification to an operator in consideration of a state of the operator is provided. A system including a work machine includes: an electroencephalograph that detects brain waves of an operator who manipulates the work machine; a notification unit that gives a notification to the operator; and a controller. The controller receives a signal indicating a detection result of the brain waves from the electroencephalograph, calculates an amplitude of the brain waves, and determines a manner of the notification by the notification unit based on a magnitude of the amplitude.

### TECHNICAL FIELD

The present disclosure relates to a system including a work machine, a control method, and a work machine.

### BACKGROUND ART

Japanese Patent Laying-Open No. 2001-067569 (PTL 1) discloses a danger warning system that gives a warning upon detection that a person has entered a dangerous area. A portable unit can be worn by a person and includes a sensor group including a brain wave sensor, a blood pressure sensor, a sweat sensor, and the like. When the person unconsciously and carelessly approaches the dangerous area, a normal warning is issued. When the person recognizes that he/she has approached the dangerous area, no warning is issued. After issuance of the normal warning, the system comprehensively assesses detection signals from the sensor group. If the system determines that the person is irritated by the warning, it controls the warning output level to be lowered so as to suppress his/her irritation.

### CITATION LIST

### Patent Literature

PTL 1: Japanese Patent Laying-Open No. 2001-067569

### SUMMARY OF INVENTION

### Technical Problem

In an existing alarm system that gives an alarm to an operator of a work machine, the same alarm is issued at all times without consideration of the degree of difficulty of work and a change in the state of the operator.

The present disclosure proposes a system including a work machine, a control method, and the work machine that allow a notification to be given to an operator in consideration of the state of the operator.

### Solution to Problem

According to an aspect of the present disclosure, a work machine or a system including a work machine is proposed. The work machine or the system includes: an electroencephalograph that detects brain waves of an operator who manipulates the work machine; a notification unit that gives a notification to the operator; and a controller. The controller receives a signal indicating a detection result of the brain waves from the electroencephalograph, calculates an amplitude of the brain waves, and determines a manner of the notification by the notification unit based on a magnitude of the amplitude.

According to an aspect of the present disclosure, a method of controlling a notification unit that gives a notification to an operator who manipulates a work machine is proposed. The method includes the following processes. A first process is to detect brain waves of the operator. A second process is to calculate an amplitude of the brain waves. A third process is to determine a manner of the notification by the notification unit based on a magnitude of the amplitude of the brain waves.

### Advantageous Effects of Invention

The system including a work machine, the control method, and the work machine according to the present disclosure allow a notification to be given to the operator in consideration of the state of the operator.

### BRIEF DESCRIPTION OF DRAWINGS

FIG. 1 is a side view schematically showing a configuration of a hydraulic excavator.

FIG. 2 is a block diagram illustrating a system configuration according to an embodiment.

FIG. 3 is a side view of a head schematically showing an arrangement of electrodes of an electroencephalograph.

FIG. 4 is a schematic diagram showing attention resource theory for humans.

FIG. 5 is a schematic diagram showing a relation between a degree of difficulty of work and an allocation of attention resources.

FIG. 6 is a graph showing brain waves appearing when stimulation is given during work with a low degree of difficulty.

FIG. 7 is a graph showing brain waves appearing when stimulation is given during work with a medium degree of difficulty.

FIG. 8 is a graph showing brain waves appearing when stimulation is given during work with a high degree of difficulty.

FIG. 9 is a graph showing a relation between the degree of difficulty of work and an amplitude of brain waves.

FIG. 10 is a flowchart illustrating a flow of a process of giving a notification to an operator.

FIG. 11 is a diagram showing a manner of a notification in association with a skill level of the operator and the degree of difficulty of work.

FIG. 12 is a schematic diagram showing an amount of attention resources during occurrence of mind wandering.

FIG. 13 is a graph showing the amplitude of brain waves appearing during occurrence of mind wandering.

FIG. 14 is a schematic diagram showing one example of issuance of an alarm based on a temporal change in the amplitude of brain waves.

FIG. 15 is a schematic diagram showing another example of issuance of an alarm based on a temporal change in the amplitude of brain waves.

### DESCRIPTION OF EMBODIMENTS

Hereinafter, embodiments will be described with reference to the accompanying drawings. In the following description, the same parts and components are denoted by the same reference characters. Their names and functions are also the same. Therefore, the detailed description thereof will not be repeated. Extraction of any configurations from the embodiment and any combination thereof are also originally intended.

### Overall Configuration of Hydraulic Excavator 1

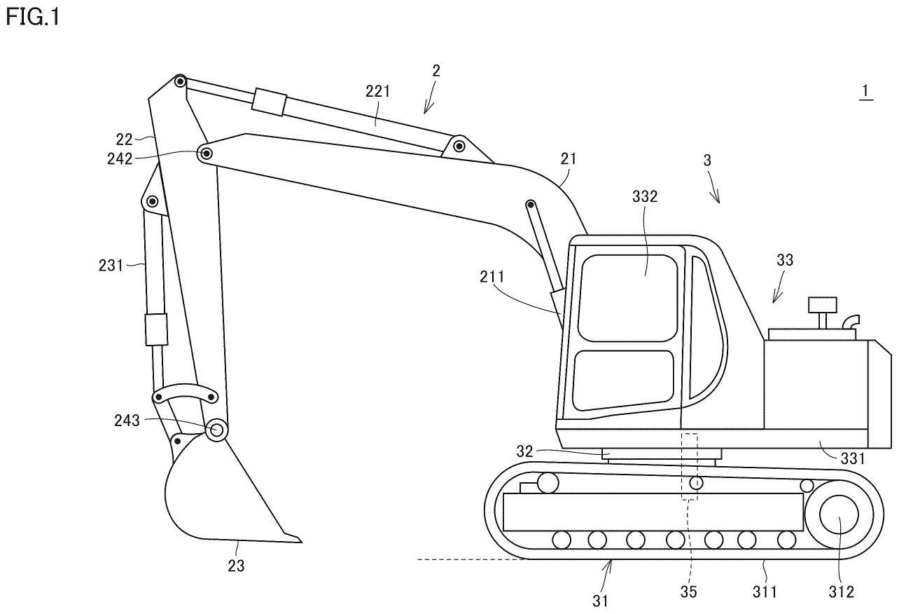

In an embodiment, a hydraulic excavator 1 will be described as an example of a work machine. FIG. 1 is a side view schematically showing a configuration of hydraulic excavator 1.

As shown in FIG. 1, hydraulic excavator 1 includes a work implement 2 and a vehicular body 3. Vehicular body 3 includes a traveling unit 31, a swing circle 32, a revolving unit 33, and a revolution motor 35.

Traveling unit 31 includes a pair of left and right crawler belt apparatuses 311 and a travel motor 312. The pair of left and right crawler belt apparatuses 311 each have a crawler belt. As travel motor 312 is driven, a pair of left and right crawler belts rotates, so that hydraulic excavator 1 is self-propelled.

Revolving unit 33 is installed on traveling unit 31 with swing circle 32 interposed therebetween. Swing circle 32 is connected to revolution motor 35. Swing circle 32 is rotated by rotational driving of revolution motor 35. As swing circle 32 rotates, revolving unit 33 revolves with respect to traveling unit 31.

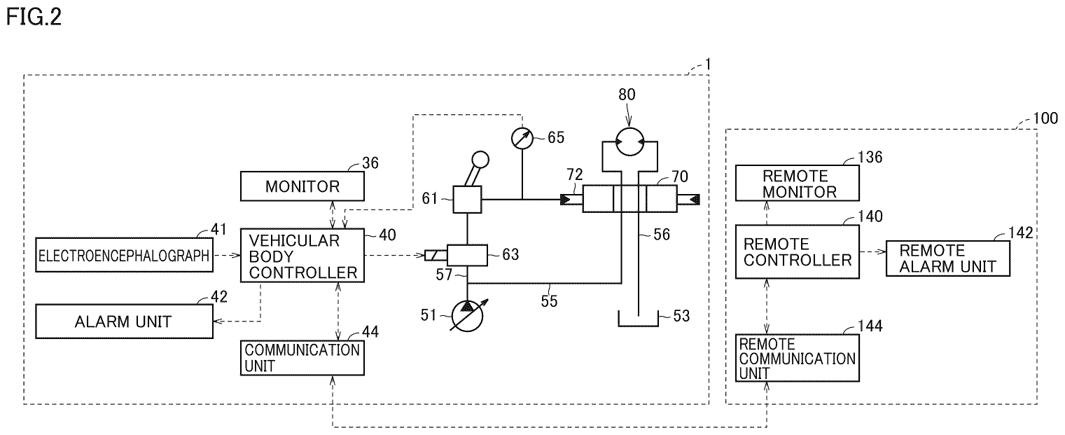

Revolving unit 33 includes: a frame 331 to which work implement 2 is attached; an operator's cab 332; and a vehicular body controller 40 (see FIG. 2) that controls the operation of hydraulic excavator 1. Operator's cab 332 is disposed, for example, on the front left side (the vehicle front side) of revolving unit 33.

Work implement 2 is supported by frame 331 on the front side of revolving unit 33 and, for example, on the right side of operator's cab 332. Work implement 2 includes a boom 21, an arm 22, a bucket 23, a boom cylinder 211, an arm cylinder 221, and a bucket cylinder 231.

Boom 21 is attached to revolving unit 33. Boom 21 has a proximal end rotatably coupled to revolving unit 33 by a boom foot pin (not shown).

Arm 22 is attached to a distal end of boom 21. Arm 22 has a proximal end rotatably coupled to the distal end of boom 21 by a boom distal end pin 242.

Bucket 23 is attached to a distal end of arm 22. Bucket 23 is rotatably coupled to the distal end of arm 22 by an arm distal end pin 243. Bucket 23 is an example of an attachment attachable to a distal end of work implement 2.

Boom 21 can be driven by boom cylinder 211. Boom cylinder 211 is driven by hydraulic oil supplied from a hydraulic pressure source. Such driving allows boom 21 to be pivotable about the boom foot pin (not shown) in an up-down direction with respect to revolving unit 33.

Arm 22 can be driven by arm cylinder 221. Arm cylinder 221 is driven by hydraulic oil supplied from the hydraulic pressure source. Such driving allows arm 22 to be pivotable about boom distal end pin 242 in the up-down direction with respect to boom 21.

Bucket 23 can be driven by bucket cylinder 231. Bucket cylinder 231 is driven by hydraulic oil supplied from the hydraulic pressure source. Such driving allows bucket 23 to be pivotable about arm distal end pin 243 in the up-down direction with respect to arm 22. Work implement 2 can be driven in this way.

### System Configuration

FIG. 2 is a block diagram illustrating a system configuration according to the embodiment. As shown in FIG. 2, hydraulic excavator 1 mainly includes vehicular body controller 40 and a hydraulic circuit extending from a hydraulic pump 51 to a hydraulic actuator 80. The solid line in FIG. 2 indicates the hydraulic circuit. The dashed line in FIG. 2 indicates an electric circuit. FIG. 2 shows only a part of the electric circuit constituting hydraulic excavator 1 in the embodiment.

Vehicular body controller 40 serves as a controller that controls the operation of hydraulic excavator 1, and includes a central processing unit (CPU), a non-volatile memory, a timer, and the like.

A monitor 36 is connected to vehicular body controller 40. Monitor 36 is disposed, for example, in operator's cab 332. Vehicular body controller 40 causes monitor 36 to display vehicular body information about hydraulic excavator 1. The vehicular body information about hydraulic excavator 1 includes, for example, an image of the surroundings of hydraulic excavator 1, a remaining amount of fuel, a temperature of a coolant, a temperature of hydraulic oil, an operation status of an air conditioner, and the like. Monitor 36 may include, for example, a mechanical monitor.

Hydraulic pump 51 supplies hydraulic oil used for the operation of work implement 2, the revolution of revolving unit 33, and the traveling operation of traveling unit 31. Hydraulic pump 51 is coupled to a drive shaft of an engine. As the rotational driving force of the engine is transmitted to hydraulic pump 51, hydraulic pump 51 is driven to discharge the hydraulic oil. Hydraulic pump 51 serves as a variable displacement hydraulic pump, for example, having a swash plate that is tilted at various angles so as to change the discharge volume.

A tank 53 stores oil used by hydraulic pump 51. As hydraulic pump 51 is driven, the oil stored in tank 53 is suctioned from tank 53 and supplied to a main valve 70 through a hydraulic oil supply path 55.

Main valve 70 is a spool type valve configured such that a rod-shaped spool is moved to switch the direction in which the hydraulic oil flows. The spool axially moves to adjust the amount of hydraulic oil to be supplied to various types of hydraulic actuators 80. Hydraulic oil is to be supplied to hydraulic actuator 80 for activating the same.

Hydraulic actuator 80 includes boom cylinder 211, arm cylinder 221, and bucket cylinder 231 shown in FIG. 1. Hydraulic actuator 80 includes travel motor 312 and revolution motor 35 shown in FIG. 1. The hydraulic oil is supplied to hydraulic actuator 80 through main valve 70. The hydraulic pressure is controlled to be supplied to and discharged from hydraulic actuator 80, to thereby control the operation of work implement 2, the revolution of revolving unit 33, and the traveling operation of traveling unit 31.

The hydraulic oil discharged from main valve 70 is returned to tank 53 through a hydraulic oil discharge path 56.

Part of oil delivered from hydraulic pump 51 is branched from hydraulic oil supply path 55 and flows into a pilot oil path 57. Part of oil delivered from hydraulic pump 51 is reduced in pressure in a self-pressure reduction valve, and the oil reduced in pressure is used as pilot oil. Pilot oil is to be supplied to main valve 70 for activating the spool of main valve 70. Main valve 70 has a pair of pressure receiving chambers 72. As the pilot oil having prescribed hydraulic pressure (pilot pressure) is supplied to each pressure receiving chamber 72, the spool moves according to the pilot pressure to thereby control main valve 70.

A manipulation device 61 is provided in pilot oil path 57. Manipulation device 61 is disposed inside operator's cab 332. Manipulation device 61 is manipulated by an operator to operate hydraulic excavator 1. The pilot pressure applied to main valve 70 is controlled by manipulation of manipulation device 61. Manipulation device 61 has a manipulation lever. The pilot oil with pressure corresponding to the amount of manipulation of the manipulation lever is output from manipulation device 61 and supplied to each pressure receiving chamber 72 of main valve 70.

Pilot oil path 57 is provided with a solenoid valve 63. Solenoid valve 63 is connected to pilot oil path 57 upstream from manipulation device 61 in the flow direction of the pilot oil. Solenoid valve 63 adjusts the pilot oil supplied to manipulation device 61 based on a control signal from vehicular body controller 40.

A branch pipe is connected to pilot oil path 57 downstream from manipulation device 61 in the flow direction of the pilot oil. This branch pipe is provided with a pressure gauge 65. Pressure gauge 65 detects the pressure of the pilot oil having passed through manipulation device 61. The detection signal of the hydraulic pressure detected by pressure gauge 65 is input into vehicular body controller 40. The detection signal of the hydraulic pressure detected by pressure gauge 65 is an example of the manipulation signal corresponding to the manipulation of manipulation device 61.

Pressure gauge 65 may be a pressure sensor that outputs an electrical signal proportional to the pilot pressure. When the pilot pressure detected by the pressure sensor is equal to or greater than a prescribed threshold value, for example, equal to or greater than 5 kg/cm², vehicular body controller 40 may determine that manipulation device 61 has been manipulated.

Pressure gauge 65 may be a pressure switch that is switched between ON and

OFF when the pilot pressure reaches prescribed pressure. The threshold value for the pressure switch may be set at 5 kg/cm², for example. In this case, when the pressure switch is switched from OFF to ON, vehicular body controller 40 may detect that pressure has occurred in pilot oil path 57, and then determine that manipulation device 61 has been manipulated.

Based on the detection result of pressure gauge 65, vehicular body controller 40 can determine whether or not hydraulic excavator 1 is performing work. When pressure gauge 65 detects a change in the pressure of the pilot oil supplied to main valve 70 that supplies the hydraulic oil to hydraulic actuator 80 in any one of boom cylinder 211, arm cylinder 221, and bucket cylinder 231, then, vehicular body controller 40 may determine that hydraulic excavator 1 is performing work with the use of work implement 2.

When pressure gauge 65 detects a change in the pressure of the pilot oil supplied to main valve 70 that supplies the hydraulic oil to travel motor 312, then, vehicular body controller 40 may determine that hydraulic excavator 1 is traveling. When pressure gauge 65 detects a change in the pressure of the pilot oil supplied to main valve 70 that supplies the hydraulic oil to revolution motor 35, then, vehicular body controller 40 may determine that hydraulic excavator 1 is revolving.

Manipulation device 61 may be an electrical manipulation device. The electrical manipulation device outputs a signal indicating the direction and the amount of manipulation of the manipulation device to vehicular body controller 40. The signal output from the electrical manipulation device is an example of a manipulation signal in accordance with the manipulation of manipulation device 61. Upon receipt of the signal from the electrical manipulation device, vehicular body controller 40 may determine that hydraulic excavator 1 is performing work.

An electroencephalograph 41 is connected to vehicular body controller 40. Electroencephalograph 41 detects brain waves of an operator who manipulates hydraulic excavator 1. The operator who is aboard operator's cab 332 is required to wear a hard hat, and electroencephalograph 41 may be attached to the inside of the hard hat. The operator who is aboard operator's cab 332 may wear a head cap equipped with electroencephalograph 41. Vehicular body controller 40 receives a signal indicating the detection result of the brain waves from electroencephalograph 41.

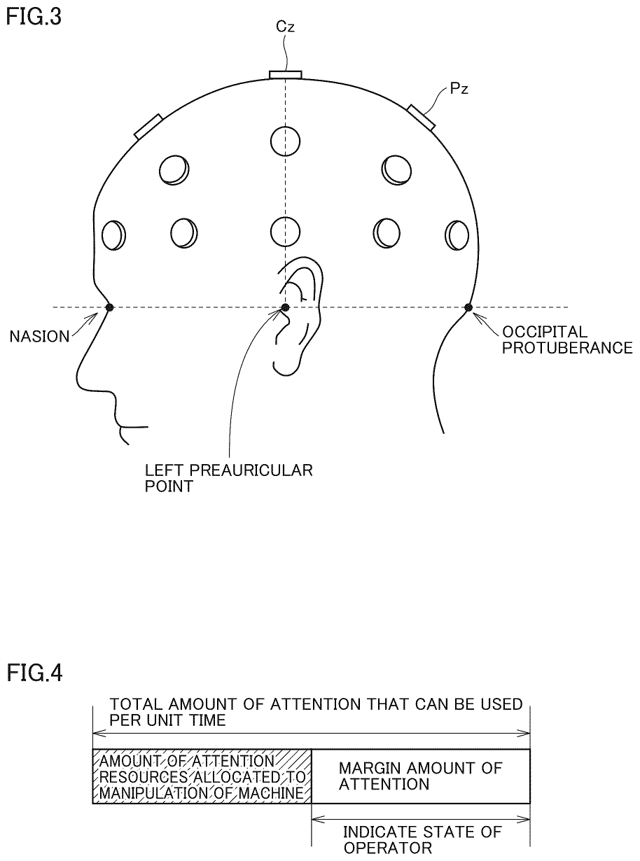

FIG. 3 is a side view of a head schematically showing an arrangement of electrodes of electroencephalograph 41. The electrodes are arranged according to the international 10-20 method. The arrangement of electrodes at the vertex (Czar) is determined by: a midpoint between a nasion and an occipital protuberance; and a midpoint between left and right preauricular points. The arrangement of electrodes at the midline parietal region (Pz) is determined by the position at which a line passing through the midline to connect Cz and the occipital protuberance is divided in a proportion of 2:3.

The present embodiment focuses on P300 among the brain waves detected by electroencephalograph 41. P300 has a peak appearing after an elapse of about 300 ms since application of stimulation. P300 is known as one example of an event-related potential. P300 is known as a biological signal related to the amount of human attention. It is known that P300 exhibits a high amplitude on the midline of the brain and exhibits a maximum amplitude particularly at Pz.

P300 is used as an indicator for grasping the state of the operator who manipulates the work machine. P300 can be used to estimate how much attention the operator manipulating the work machine is paying to the manipulation. For example, the dual-task technique allows such estimation. In the dual-task technique, during execution of a main task, a sub-task different from the main task is given. Based on the magnitude of the amplitude of P300 appearing when the sub-task is given, the amount of attention allocated to the sub-task can be estimated. The main task is, for example, to manipulate hydraulic excavator 1. As the sub-task used herein, for example, tactile stimulation is given to a fingertip of the operator with a degree of strength not to interfere with the manipulation of hydraulic excavator 1. The tactile stimulation may be, for example, electrical stimulation, vibration, and the like.

Referring back to FIG. 2, vehicular body controller 40 having received the detection result of the brain waves from electroencephalograph 41 filters the raw data of the brain waves to extract P300 in real time. Vehicular body controller 40 monitors the state of P300, specifically, the magnitude of the amplitude of P300. Vehicular body controller 40 analyzes the state of the operator in real time based on the state of P300 and uses the analysis result to monitor the timing to give a notification to the operator.

An alarm unit 42 is connected to vehicular body controller 40. Alarm unit 42 issues an alarm for giving a notification to the operator. An auditory notification such as an alarm sound, voice, or stereoscopic sound may be issued. The loudness level, the length, and the like of the sound may be changeable. A tactile notification such as vibration may also be given. Monitor 36 connected to vehicular body controller 40 may provide a visual notification for giving a notification to the operator. Monitor 36 may include, for example, a head-up display. Monitor 36 and alarm unit 42 correspond to one example of a notification unit that gives a notification to the operator.

A communication unit 44 is connected to vehicular body controller 40. Communication unit 44 communicates with a base station 100 external to hydraulic excavator 1. Base station 100 is equipped with a remote controller 140, a remote monitor 136, a remote alarm unit 142, and a remote communication unit 144. Communication unit 44 of hydraulic excavator 1 communicates with remote communication unit 144 of base station 100 via a network. The network may include at least one of the Internet, a local area network (LAN), a cellular phone communication network, and a satellite communication network.

For example, vehicular body controller 40 generates signals indicating: information to be displayed on remote monitor 136 of base station 100; and an alarm to be issued from remote alarm unit 142 of base station 100. Communication unit 44 transmits the signals generated by vehicular body controller 40 to remote communication unit 144. Remote controller 140 receives the signals generated by vehicular body controller 40 via remote communication unit 144. Remote controller 140 causes remote monitor 136 to display information and causes remote alarm unit 142 to issue an alarm. Remote alarm unit 142 gives a notification to a person inside base station 100, for example, an administrator of the operator who manipulates hydraulic excavator 1. The administrator having received the notification can grasp the state of the operator in real time.

### Attention Resource Theory

FIG. 4 is a schematic diagram showing attention resource theory for humans. The attention resource theory for humans is a hypothesis theory that the total amount of attention that can be used per unit time is fixed for each human. As shown in FIG. 4, when the operator is manipulating the work machine, a part of a fixed total amount of attention resources is allocated to the manipulation of the work machine. Among the fixed total amount of attention resources, the amount of attention resources that are not allocated to the manipulation of the work machine but can be allocated to other operations/manipulations is referred to as a margin amount of attention. This margin amount of attention represents the state of the operator.

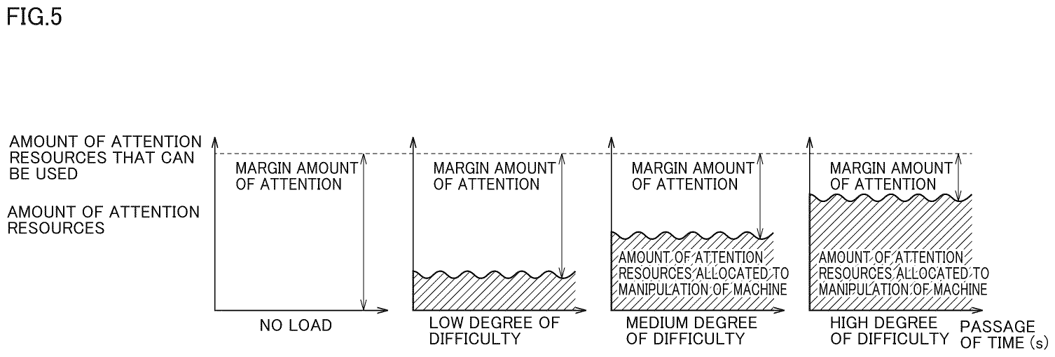

FIG. 5 is a schematic diagram showing the relation between the degree of difficulty of work and the allocation of attention resources. FIG. 5 shows the amount of attention resources allocated to the manipulation of the work machine and the margin amount of attention in each of the states in which: no load is applied, i.e., work is not performed using a work machine; work with a low degree of difficulty is performed using the work machine; work with a medium degree of difficulty is performed using the work machine; and work with a high degree of difficulty is performed using the work machine.

As described above, the amount of attention resources that can be used by the operator is fixed. Irrespective of the degree of difficulty of the work, the total amount of attention resources is fixed. A lower degree of difficulty of the work leads to a smaller amount of attention resources allocated to the manipulation of the work machine. The margin amount of attention increases accordingly. In contrast, a higher degree of difficulty of the work leads to a larger amount of attention resources allocated to the manipulation of the work machine. The margin amount of attention decreases accordingly.

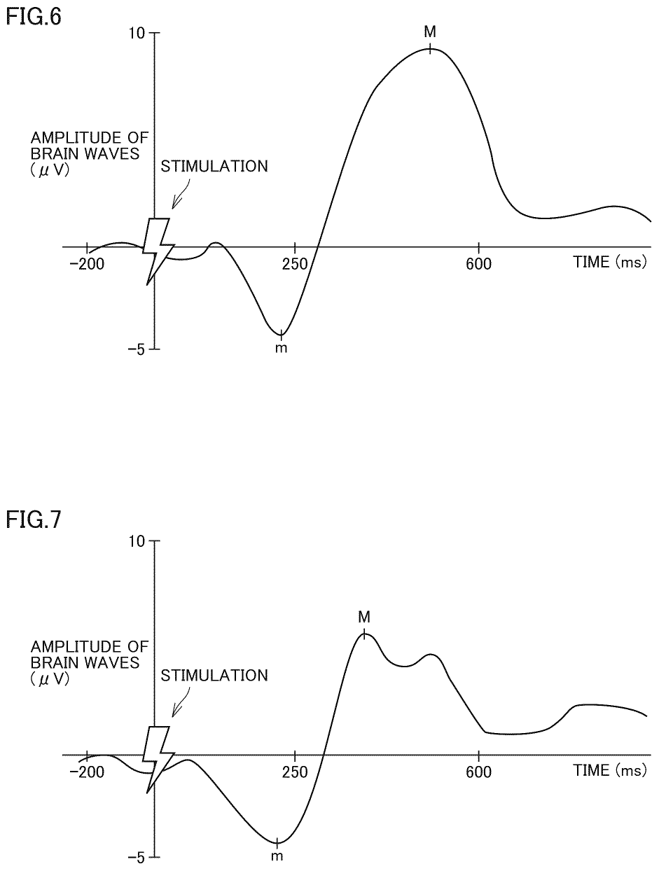

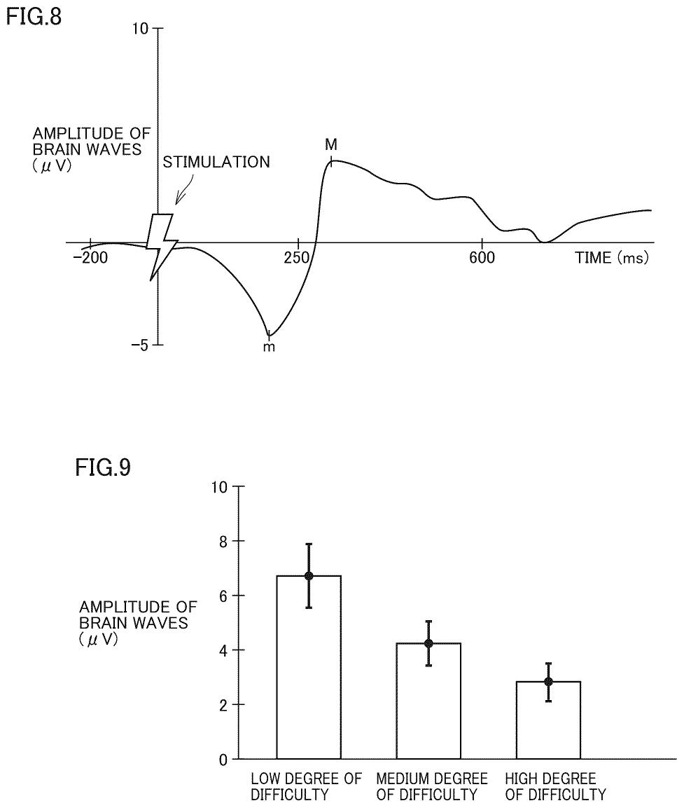

FIG. 6 is a graph showing how the brain waves of the operator appear over passage of time when electric stimulation is given to the operator who is manipulating the work machine to perform work with a low degree of difficulty. FIG. 7 is a graph showing how the brain waves of the operator appear over passage of time when electric stimulation is given to the operator who is manipulating the work machine to perform work with a medium degree of difficulty. FIG. 8 is a graph showing how the brain waves of the operator appear over passage of time when electric stimulation is given to the operator who is manipulating the work machine to perform work with a high degree of difficulty. The horizontal axis in each of FIGS. 6 to 8 indicates the passage of time. The vertical axis in each of FIGS. 6 to 8 indicates the amplitude of brain waves.

In FIGS. 6 to 8, P300 is detected. At time 0, electrical stimulation is given to the operator. P300 exhibits a local minimum value m before time 250 ms and exhibits a local maximum value M after time 300 ms. The difference between local maximum value M and local minimum value m shows the magnitude of the amplitude of the brain waves (P300), and shows an indicator indicating how much the operator's attention has been allocated to the given electrical stimulation. As more attention is allocated to the electrical stimulation, the amplitude of P300 is larger.

FIG. 9 is a graph showing the relation between the degree of difficulty of work and the amplitude of brain waves. The vertical axis in FIG. 9 indicates the amplitude of brain waves. FIG. 9 shows an average of the amplitudes of P300s calculated for a plurality of operators. Error bars shown in FIG. 9 indicate standard errors.

As shown in FIGS. 6 to 9, the amplitude of P300 is large during work with a low degree of difficulty, is smaller during work with a medium degree of difficulty than during work with a low degree of difficulty, and is further smaller during work with a high degree of difficulty. A lower degree of difficulty of the manipulation of the work machine leads to a larger amplitude of P300. It is considered that this is because, as shown in FIG. 5, the margin amount of attention is large during work with a low degree of difficulty of the manipulation of the work machine, and the margin amount of attention is small during work with a high degree of difficulty of the manipulation of the work machine.

As shown in FIGS. 6 and 9, when the operator has a sufficient margin amount of attention due to the work with a low degree of difficulty, the brain can sufficiently respond to the electrical stimulation, which leads to a larger amplitude of P300. As shown in FIGS. 8 and 9, during work with a high degree of difficulty, the operator's attention is directed to execution of difficult work, and more attention resources of the operator are allocated to the manipulation of the work machine. Thus, the brain waves are less likely to appear in response to the electrical stimulation. As the brain cannot respond to electrical stimulation, the amplitude of P300 becomes small.

Therefore, by acquiring the amplitude of P300 of the operator who manipulates the work machine during the work by the work machine, the state of the operator (the margin amount of attention) can be estimated.

The degree of difficulty of the work is different for each operator. Even when an inexperienced operator feels that the degree of difficulty of the work is high, a skilled operator may feel that the degree of difficulty of the same work is low since such a skilled operator is accustomed to performing the work. The margin amount of attention paid during execution of prescribed work may be different between an inexperienced operator and a skilled operator. The margin amount of attention paid during execution of prescribed work may be small in the case of an inexperienced operator and may be large in the case of a skilled operator. By acquiring the amplitude of P300 of each operator during the work, the margin amount of attention of each operator can be estimated.

The margin amount of attention may be different depending on the physical and mental health conditions of the operator. When there is a concern about the health condition, attention resources are allocated to the concern about the health condition, so that the amount of attention resources that can be allocated to other issues decreases, and thus, the margin amount of attention decreases. If there is no concern about the health condition, the margin amount of attention increases.

### Control for Notification to Operator

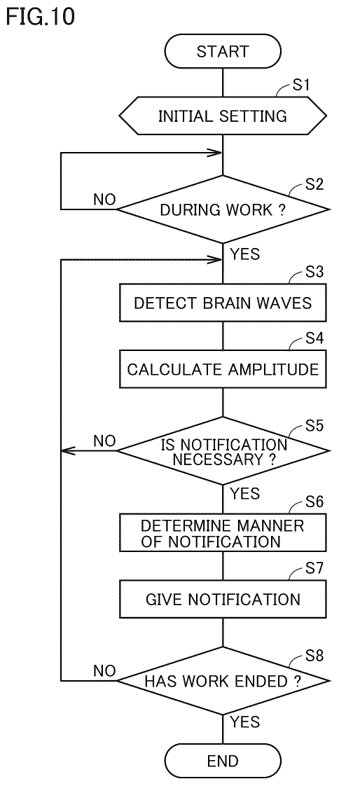

The following describes the control for giving a notification to an operator who manipulates the work machine, based on the brain waves of the operator. FIG. 10 is a flowchart illustrating a flow of a process of giving a notification to the operator.

The degree of difficulty felt for certain work (whether the work is felt difficult (a high degree of difficulty) or felt easy (a low degree of difficulty)) is different for each operator. The margin amount of attention paid during prescribed work is different for each operator. The margin amount of attention is different also depending on the health condition of the operator. Thus, in order to adjust the system to the characteristics of each individual, an initial setting is made in step S1.

For example, before starting the work, an operator who is aboard operator's cab 332 may perform an N-back task using monitor 36 disposed in operator's cab 332. The N-back task is a cognitive task for which the degree of difficulty can be set in a stepwise manner. As the N-back task, a task may be performed to see characters displayed on monitor 36 at constant time intervals to check whether or not the currently displayed characters are the same as those having been displayed N times before. During the execution of the N-back task, electrical stimulation is given to the operator's finger with a degree of strength not to interfere with the task. A larger margin amount of attention resources allows more attention resources to be allocated to electrical stimulation, which leads to a larger brain response to the electrical stimulation (P300). P300 allows estimation of the margin amount of the attention resources. Electroencephalograph 41 detects the brain waves of the operator who is performing the N-back task. Vehicular body controller 40 extracts P300 from the brain waves of the operator and calculates the amplitude of P300. Based on the magnitude of the amplitude of P300, vehicular body controller 40 determines a threshold value for issuance of an alarm sound.

The operator may perform the N-back task before the operator gets into operator's cab 332. For example, the operator may use the application of a smartphone to perform the N-back task in advance. The operator transmits the detection result of the P300 obtained during the N-back task performed in advance or the amplitude of the P300 calculated based on the detection result of the P300 to vehicular body controller 40. Vehicular body controller 40 determines the threshold value for issuance of an alarm sound based on the magnitude of the amplitude of the P300.

In place of the N-back task, an easy manipulation of the work machine may be performed to measure the P300 of the operator during this manipulation. The operator who is aboard operator's cab 332 performs a prescribed manipulation of the work machine in several patterns, and electroencephalograph 41 detects P300 of the operator who is performing the manipulation. Vehicular body controller 40 calculates the amplitude of P300. Vehicular body controller 40 determines a threshold value for issuance of an alarm sound based on the magnitude of the amplitude of the P300.

After getting into operator's cab 332 and before starting the work, the operator puts on electroencephalograph 41 for detecting brain waves and an external stimulator for giving tactile stimulation. The external stimulator gives tactile stimulation such as electrical stimulation or vibrations to the operator.

After the initial setting of the system, in step S2, vehicular body controller 40 determines whether or not the work machine is performing the work. As described with reference to FIG. 2, vehicular body controller 40 receives an input of a manipulation signal corresponding to the manipulation of manipulation device 61 from pressure gauge 65 or manipulation device 61 and thereby can determine that hydraulic excavator 1 is performing the work. The work used herein includes revolution of revolving unit 33 and traveling of hydraulic excavator 1 by traveling unit 31, in addition to the work using work implement 2.

When it is determined that the work machine is not performing the work (NO in step S2), the determination in step S2 is repeated. When it is determined that the work machine is performing the work (YES in step S2), the process proceeds to step S3. Then, electroencephalograph 41 detects the brain waves of the operator that appear when tactile stimulation is given to the operator who is manipulating the work machine. Vehicular body controller 40 receives a signal indicating the detection result of the brain waves from electroencephalograph 41.

In step S4, vehicular body controller 40 extracts P300 by filtering the detection result of the brain waves input from electroencephalograph 41. Vehicular body controller 40 calculates the amplitude of the P300.

In step S5, vehicular body controller 40 determines whether or not a notification needs to be given to the operator. For example, it is determined that a notification needs to be given to the operator of the work machine in the case where a person is approaching the work machine or there is a possibility that the work machine may come into contact with an obstacle if the work machine continues to perform the work. The situation around the work machine can be acquired by a sensor mounted on the work machine or disposed outside the work machine. The sensor may be an imaging device that captures an image of an imaging target. The imaging device may be disposed at a prescribed point in a worksite or may be mounted on a drone.

When it is determined that a notification is necessary (YES in step S5), the process proceeds to step S6, in which vehicular body controller 40 determines a manner of notification to the operator by the notification unit (monitor 36 and alarm unit 42).

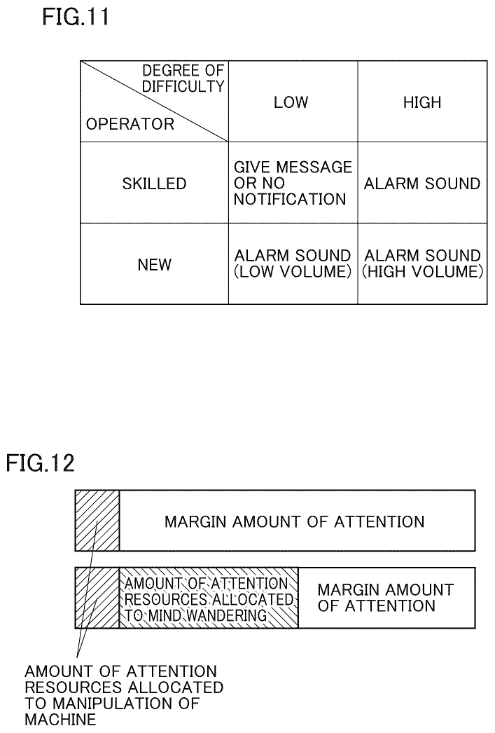

FIG. 11 is a diagram showing a manner of notification in association with the skill level of the operator and the degree of difficulty of work. FIG. 11 shows an appropriate type of notification to be given to a skilled operator or a new operator when such a skilled or new operator is performing work with a low degree of difficulty or a high degree of difficulty. The skill level of the operator is determined by the initial setting in step S1. The degree of difficulty of the work is determined based on the magnitude of the amplitude of the P300 calculated in step S4. When the amplitude of the P300 is large, the degree of difficulty of the work is determined as low. When the amplitude of the P300 is small, the degree of difficulty of the work is determined as high.

While a skilled operator is performing the work with a low degree of difficulty, the margin amount of attention is large. Accordingly, even if a complicated notification is given, it can be expected that the skilled operator will recognize and understand the details of the notification. Thus, instead of simply issuing a warning sound, an audio message notifying the operator about the situation or the instruction can be given to the operator. Alternatively, if the margin amount of attention of the skilled operator is large, it is expected that the attention resources can be allocated in order for the skilled operator himself/herself to grasp the situation around the work machine. Thus, giving no notification makes it possible to avoid a situation that the operator feels annoyance at a notification.

While the skilled operator is performing the work with a high degree of difficulty, the margin amount of attention is small Accordingly, if a complicated notification is given, the skilled operator cannot understand the details of the notification, with the result that the notification may become meaningless as an alarm. Thus, giving a notification with a warning sound makes it possible to reliably arouse the operator's attention.

While an inexperienced new operator is performing work, giving a complicated notification may prevent this operator from understanding the details of the notification, with the result that this notification may become meaningless as an alarm. Accordingly, a notification is given with a warning sound irrespective of the degree of difficulty of the work. During execution of the work with a high degree of difficulty, issuing a warning sound with a larger volume makes it possible to reliably arouse the operator's attention.

Thus, the degree of difficulty of work that is felt by the operator is determined based on the magnitude of the amplitude of P300. Then, based on the determined degree of difficulty of the work, the manner of notification to be given by the notification unit is determined. Thereby, an appropriate notification can be given to the operator according to the operator's skill level. Since a notification can be given to the operator in consideration of the state of the operator, the operator having received the notification can more reliably recognize, for example, that an obstacle hindering the work of the work machine exists around the work machine.

Referring back to FIG. 10, in step S7, vehicular body controller 40 outputs, to alarm unit 42, a signal giving an instruction to give a notification in the manner of notification determined in step S6. Alarm unit 42 having received the signal issues an alarm to give a notification to the operator.

In step S8, vehicular body controller 40 determines whether or not the work has ended. When the input of the manipulation signal corresponding to the manipulation of manipulation device 61 from pressure gauge 65 or manipulation device 61 continues, vehicular body controller 40 can determine that the work has not ended. When the input of the manipulation signal corresponding to the manipulation of manipulation device 61 from pressure gauge 65 or manipulation device 61 is stopped, vehicular body controller 40 can determine that the work has ended.

When it is determined in step S5 that no notification is necessary (NO in step S5), and when it is determined in step S8 that the work has not ended (NO in step S8), the process returns to step S3. Then, the detection of the operator's brain waves and the calculation of the amplitude of P300 are repeated.

When it is determined in step S8 that the work has ended (YES in step S8), the process ends (“END” in FIG. 10).

### Control for Notification During Mind Wandering

During the work, a phenomenon may occur in which the operator's attention is scattered since the operator's attention is distracted from the manipulation of the work machine and the operator thinks about the issue not related to the work (mind wandering). As described above, while the operator is performing the work with a low degree of difficulty, a small amount of attention resources is allocated to the manipulation of the work machine. During the work with a high degree of difficulty, a larger amount of attention resources is allocated to the manipulation of the work machine, and thus, mind wandering is less likely to occur. Mind wandering is more likely to occur during the work with a low degree of difficulty.

FIG. 12 is a schematic diagram showing the amount of attention resources during occurrence of mind wandering. The horizontal bars shown in FIG. 12 indicate a total amount of attention as in FIG. 4. The upper part in FIG. 12 shows the allocation of the amount of attention resources that is given when mind wandering does not occur during the work with a low degree of difficulty. Due to the work with a low degree of difficulty, the amount of attention resources allocated to the manipulation of the work machine is relatively small while the margin amount of attention is large. Thus, if mind wandering does not occur, the amplitude of P300 becomes large.

The lower part in FIG. 12 shows the allocation of the amount of attention resources that is given when mind wandering occurs during the work with a low degree of difficulty. The amount of attention resources allocated to the manipulation of the work machine is the same as that allocated when mind wandering does not occur. Since a part of the amount of attention resources is allocated to the mind wandering, the margin amount of attention is smaller than that obtained when mind wandering does not occur.

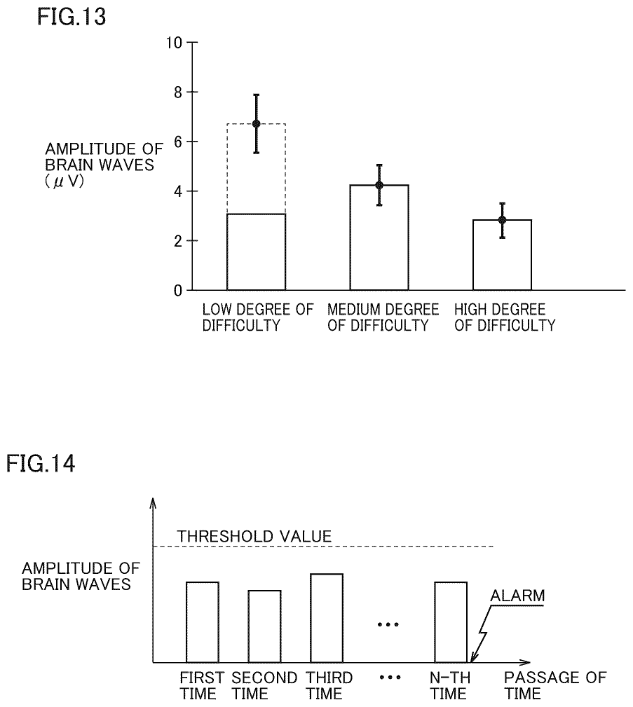

FIG. 13 is a graph showing the amplitude of brain waves appearing during occurrence of mind wandering. As in FIG. 9, the vertical axis in FIG. 13 indicates the amplitude of brain waves. In the amplitude of brain waves appearing during the work with a low degree of difficulty, the dashed line indicates the magnitude of the amplitude of P300 appearing when mind wandering does not occur, as shown also in FIG. 9. The solid line indicates the magnitude of the amplitude of the P300 appearing during occurrence of mind wandering. Since the amount of attention resources is allocated to the mind wandering, the margin amount of attention decreases accordingly. As a result, the magnitude of the amplitude of P300 becomes smaller when mind wandering occurs.

Based on the temporal change in the magnitude of the amplitude of P300, vehicular body controller 40 can determine that mind wandering has occurred. Electroencephalograph 41 detects the brain waves occurring in the operator who is manipulating the work machine when tactile stimulation is given to this operator. As described with reference to FIGS. 6 to 8, when tactile stimulation is given, the amplitude of P300 responding to this tactile stimulation can be calculated. When tactile stimulation is given multiple times at random time intervals, the amplitude of each P300 responding to each tactile stimulation can be calculated. When the magnitudes of the amplitudes of the plurality P300s obtained upon application of multiple tactile stimulations are arranged in a time series manner, a temporal change in the magnitude of the amplitude of the P300 can be grasped. Based on the temporal change in the magnitude of the amplitude of the P300, vehicular body controller 40 can determine whether or not to give a notification to the operator. The determination as to whether or not to give a notification to the operator corresponds to one example of the determination about the manner of notification.

FIG. 14 is a schematic diagram showing one example of issuance of an alarm based on a temporal change in the amplitude of brain waves. As shown in FIG. 14, a threshold value is set for the magnitude of the amplitude of P300. The threshold value is defined as a value that is different for each operator. This threshold value can be determined by the initial setting in step S1 described above.

FIG. 14 shows the amplitudes of P300s obtained in response to the first tactile stimulation to the N-th tactile stimulation. During a prescribed time period, i.e., during a time period from when the first tactile stimulation is given to when the N-th tactile stimulation is given, the magnitude of the amplitude of each P300 does not exceed the threshold value and is kept small for a prescribed time period.

In such a case, vehicular body controller 40 causes the notification unit to give a notification to the operator. If the amplitude of each P300 is kept small despite a low degree of difficulty of the currently performed work, it can be determined that mind wandering occurs. In such a case, giving a notification to the operator makes it possible to urge the operator to stop the mind wandering to concentrate on manipulating the work machine.

Further, as shown in FIGS. 8 and 9, the amplitude of the P300 becomes small also when the operator performs work with a high degree of difficulty. There is a possibility that the work with a high degree of difficulty may continue while the amplitude of the P300 is kept small. Giving a notification to the operator makes it possible to trigger the operator to notice whether or not the operator has fatigue without realizing it due to a continuous operation of the work with a high degree of difficulty for a prescribed time period.

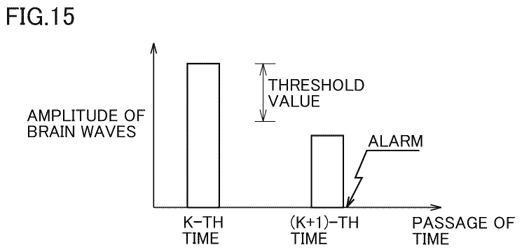

FIG. 15 is a schematic diagram showing another example of issuance of an alarm based on a temporal change in the amplitude of brain waves. FIG. 15 shows that the magnitude of the amplitude of P300 obtained in response to the (K+1)-th tactile stimulation sharply decreases as compared with the magnitude of the amplitude of P300 obtained in response to the K-th tactile stimulation. A threshold value is set for the value to which the amplitude of P300 increases or decreases. The magnitude of the amplitude of P300 detected upon application of the (K+1)-th tactile stimulation decreases by a threshold value or more as compared with the magnitude of the amplitude of P300 detected upon application of the K-th tactile stimulation.

In such a case, vehicular body controller 40 causes the notification unit to give a notification to the operator. It can be determined that the reason why the magnitude of the amplitude of P300 has decreased by a prescribed amount or more since the previous detection of P300 is because, due to occurrence of mind wandering, the amount of attention resources has been allocated to the mind wandering, and thus, the margin amount of attention has decreased. In this case, giving a notification to the operator makes it possible to urge the operator to stop the mind wandering to concentrate on manipulating the work machine.

By giving a notification to the operator based on the temporal change in the amplitude of P300 of the operator during the manipulation of the work machine, the mind wandering can be stopped at an early stage if the mind wandering occurs in the operator. By increasing the margin amount of the operator's attention, for example, the operator can pay more attention to the situation around the work machine. For example, the operator can more quickly and more reliably recognize that an obstacle that will hinder the work of the work machine exists around the work machine.

In the example described in the above embodiment, vehicular body controller 40 that controls the operation of hydraulic excavator 1 determines the manner of notification to the operator. The controller that determines the manner of notification to the operator does not necessarily have to be mounted on hydraulic excavator 1.

A system may be configured such that a controller mounted on hydraulic excavator 1 performs control to transmit a detection result of brain waves acquired by electroencephalograph 41 to an external controller, and the external controller having received the detection result of the brain waves performs control to calculate the amplitude of the brain waves to determine the manner of notification based on the magnitude of the amplitude of the brain waves. Alternatively, a system may be configured such that a controller mounted on hydraulic excavator 1 performs control to calculate the amplitude of the brain waves based on the detection result of the brain waves and transmit the calculated amplitude of the brain waves to an external controller, and the external controller having received the calculated amplitude of the brain waves performs control to determine the manner of notification based on the magnitude of the amplitude of the brain waves.

The external controller may be disposed in a worksite of hydraulic excavator 1 or may be disposed at a remote location away from the worksite of hydraulic excavator 1. For example, the external controller may be disposed in base station 100 shown in FIG. 2. The external controller may be remote controller 140 shown in FIG. 2.

In the example described in the embodiment, hydraulic excavator 1 is a manned vehicle including operator's cab 332 in which the operator is seated. Hydraulic excavator 1 may be an unmanned vehicle. Hydraulic excavator 1 may not include operator's cab 332 in which the operator is seated and manipulates hydraulic excavator 1. Hydraulic excavator 1 may not have a steering function executed by an operator who is on board. Hydraulic excavator 1 may be a work machine manipulated only by a remote control operation. The manipulation of hydraulic excavator 1 may be performed by a wireless signal from a remote control device.

A system is constructed such that, when hydraulic excavator 1 is remotely controlled, an electroencephalograph detects brain waves of an operator who manipulates the remote control device, and a notification unit gives a notification to the operator who manipulates the remote control device. The electroencephalograph that detects the brain waves of the operator may not necessarily have to be mounted on hydraulic excavator 1. The notification unit that gives a notification to the operator does not necessarily have to be mounted on hydraulic excavator 1. In the case where hydraulic excavator 1 is remotely controlled, it is also desirable to install a controller at a position where the remote control device external to hydraulic excavator 1 is installed, the controller being configured to receive a signal indicating the detection result of brain waves from the electroencephalograph, calculate the amplitude of the brain waves, and determine the manner of notification by the notification unit based on the magnitude of the amplitude.

In the embodiment, hydraulic excavator 1 has been described as an example of the work machine, but the idea of the present disclosure may be applied not only to hydraulic excavator 1 but also to other types of work machines such as a bulldozer, a wheel loader, and a dump truck.

While the embodiments have been described above, it should be understood that the embodiments disclosed herein are illustrative and non-restrictive in every respect. The scope of the present invention is defined by the terms of the claims, rather than the above description, and is intended to include any modifications within the meaning and scope equivalent to the terms of the claims.

### REFERENCE SIGNS LIST

1 hydraulic excavator, 2 work implement, 3 vehicular body, 31 traveling unit, 33 revolving unit, 35 revolution motor, 36 monitor, 40 vehicular body controller, 41 electroencephalograph, 42 alarm unit, 44 communication unit, 61 manipulation device, 65 pressure gauge, 80 hydraulic actuator, 100 base station, 136 remote monitor, 140 remote controller, 142 remote alarm unit, 144 remote communication unit, 312 travel motor, M local maximum value, m local minimum value.

1. A system including a work machine, the system comprising:
an electroencephalograph that detects brain waves of an operator who manipulates the work machine;
a notification unit that gives a notification to the operator; and
a controller, wherein
the controller receives a signal indicating a detection result of the brain waves from the electroencephalograph, calculates an amplitude of the brain waves, and determines a manner of the notification by the notification unit based on a magnitude of the amplitude.

2. The system according to claim 1, wherein the controller determines a manner of the notification based on a temporal change in the magnitude of the amplitude.

3. The system according to claim 2, wherein
a threshold value is set for the magnitude of the amplitude, and
the controller determines the manner of the notification based on a state in which the magnitude of the amplitude does not exceed the threshold value for a prescribed time period.

4. The system according to claim 2, wherein the controller determines the manner of the notification based on a state in which the magnitude of the amplitude has decreased by a prescribed amount or more since a previous detection of the brain waves.

5. The system according to claim 1, wherein the work machine includes a communication unit that communicates with an external base station.

6. The system according to claim 1, wherein the controller determines whether or not the work machine is performing work.

7. A method of controlling a notification unit that gives a notification to an operator who manipulates a work machine, the method comprising:
detecting brain waves of the operator;
calculating an amplitude of the brain waves; and
determining a manner of the notification by the notification unit based on a magnitude of the amplitude.

8. A work machine comprising:
an electroencephalograph that detects brain waves of an operator who manipulates the work machine;
a notification unit that gives a notification to the operator; and
a controller, wherein
the controller receives a signal indicating a detection result of the brain waves from the electroencephalograph, calculates an amplitude of the brain waves, and determines a manner of the notification by the notification unit based on a magnitude of the amplitude.
